(19) United States
(12) Patent Application Publication
(10) Pub. No.: US 2019/0151234 A1
(43) Pub. Date: May 23, 2019

(54) STABLE LIQUID PHARMACEUTICAL FORMULATIONS OF BENDAMUSTINE

(71) Applicant: LEIUTIS PHARMACEUTICALS PVT. LTD., Hyderabad (IN)

(72) Inventors: Kocherlakota CHANDRASHEKHAR, Secunderabad (IN); Banda NAGARAJU, Hyderabad (IN)

(73) Assignee: Leiutis Pharmaceuticals Pvt. Ltd., Hyderabad (IN)

(21) Appl. No.: 16/091,243

(22) PCT Filed: Mar. 30, 2017
(86) PCT No.: PCT/IB2017/051820

§ 371 Date: Oct. 4, 2018

(30) Foreign Application Priority Data
Apr. 5, 2016 (IN) 201641011969

(51) Int. Cl. A61K 9/00; A61K 31/4184; A61K 47/18; A61K 47/10; A61K 9/08

(52) U.S. Cl. CPC A61K 9/0019; A61K 31/4184; A61K 9/08; A61K 47/10; A61K 47/183

(57) ABSTRACT

The present invention relates to stable liquid pharmaceutical formulation of Bendamustine or a pharmaceutically acceptable salt thereof; wherein the formulation is devoid of propylene glycol.

### BACKGROUND OF THE INVENTION

Bendamustine was found useful in treating chronic lymphocytic leukemia, Hodgkin's disease, non-Hodgkin's lymphoma, multiple myeloma and lung cancer. Bendamustine hydrochloride has the following structure:

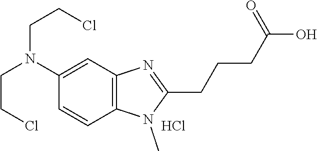 [SMILES: Cn1c(CCCC(=O)O)nc2cc(N(CCCl)CCCl)ccc21 ; Cl]

In U. S Bendamustine is available as powder for IV infusion and as solution for IV infusion under the tradename Treanda®.

Treanda® powder for IV infusion is supplied as a sterile non-pyrogenic white to off-white lyophilized powder in a single-use vial in 100 mg and 25 mg strengths. Each vial contains Bendamustine HCl and mannitol. The pH of the reconstituted solution is 2.5-3.5.

Treanda® solution is supplied in single-use vials containing either 45 mg/0.5 mL or 180 mg/2 mL of Bendamustine HCl. Each 0.5 mL vial contains 45 mg of Bendamustine hydrochloride, 162 mg of propylene glycol, and 293 mg of N,N-dimethylacetamide. The injection is intended for intravenous infusion only after dilution with either 0.9% sodium chloride injection, USP, or 2.5% dextrose/0.45% sodium chloride injection, USP. It is supplied as a sterile clear colorless to yellow solution in single-use vials at a concentration of 90 mg/mL of Bendamustine HCl.

The reconstitution of Bendamustine lyophilized powder is time consuming and cumbersome. Moreover, lyophilization of solids on a commercial scale requires specialized equipment and incurs significant expense. Because of the patient-specific dosing, repeated administration, and a dosage regimen of six 28-day cycles, there is a strong need for liquid formulations with enhanced stability. Unfortunately, while various attempts have been made to stabilize Bendamustine, all or almost all of them suffer from several disadvantages.

U.S. Pat. Nos. 8,609,707, 9,265,831 to Palepu et al., point out that stability of Bendamustine in aqueous solutions is very minimal and is limited to few hours and is therefore, not suitable for long-term storage in liquid form. Palepu explains that liquid Bendamustine composition comprising non-aqueous solvents such as polyethylene glycol, propylene glycol showed improved long term stability when compared to currently available formulations.

In still further known attempts, Bendamustine is combined with first charged cyclopolysaccharide and a stabilizing agent (which is a second charged cyclopolysaccharide) with a charge opposite to that of the first cyclopolysaccharide as discussed in WO 2012/127277 and US 2010/0216858.

US 2011/0015245 teaches use of various amphiphilic cationic compositions to stabilize Bendamustine. WO 2011/005714 teaches liposomal formulations encapsulating Bendamustine.

In another approach reported in Pharmazie (1994; 49, 10:775-777), stability of Bendamustine HCl (0.25 mg/ml in 0.9% sodium chloride) in water was studied, wherein it was found that Bendamustine was stable at 4° C. for 5 days and at 23° C. for 9 hours. While the temperature had adverse effects on stability, moderate concentrations of chloride ions increased the stability to at least some degree. However, such compositions were not stable over extended periods.

U.S. Pat. No. 8,344,006 describes liquid formulations comprising Bendamustine or pharmaceutically acceptable salts thereof, and polar aprotic solvents like dimethylacetamide and propylene glycol. Even though propylene glycol is considered to be harmless, in high concentrations it causes lactic acidosis and hyperosmolarity, hemolysis, renal toxicity including tubular dysfunction and acute tubular necrosis. When propylene glycol is present in high doses in intravenous formulations, it increases the formulation osmolality. Hence its use in patients of diminished renal function, should be monitored by determining plasmatic osmolality daily. Further, solutions of Bendamustine in propylene glycol degrade to form impurities identified as propylene glycol esters of Bendamustine. Upon exposure to an alkylene glycol, for example, propylene glycol, esters of Bendamustine can form, e.g., PG-1 and PG-2 depicted below.

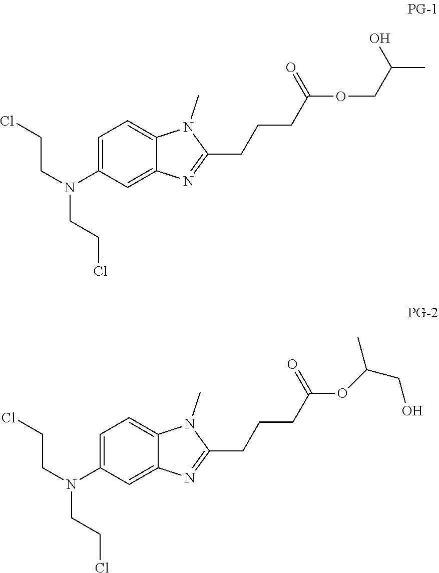 [SMILES: CC(O)COC(=O)CCCc1nc2cc(N(CCCl)CCCl)ccc2n1C ; CC(CO)OC(=O)CCCc1nc2cc(N(CCCl)CCCl)ccc2n1C]

The 90 mg/mL non-aqueous formulation of liquid Bendamustine HCl described in U.S. Pat. No. 8,344,006 exhibits bendamustine-propylene glycol ester degradation products following 12 months of storage at 5° C. These esters are present at a level greater than 1% which may not be advisable for the patient.

Hence there is a need for liquid formulations having enhanced stability. The present invention addresses this need.

### SUMMARY OF THE INVENTION

The present invention relates to liquid formulations of Bendamustine, wherein the formulation is free of propylene glycol.

Another aspect of the present invention is to provide liquid pharmaceutical formulation comprising Bendamustine, amino acids, suitable solvent or mixture of solvents and other pharmaceutically acceptable excipients.

Yet another aspect of the present invention is to provide liquid pharmaceutical formulation comprising Bendamustine, amino acids, suitable solvents such as polyethylene glycol, glycerol, water, ethanol and other pharmaceutically acceptable excipients; wherein the formulation is free of propylene glycol.

### DETAILED DESCRIPTION OF THE INVENTION

In the context of this invention “Bendamustine” refers to the pharmaceutically acceptable salts, solvates, hydrates, acids and free base forms, preferably Bendamustine hydrochloride.

As used herein, “liquid” formulations refers to formulations that contain Bendamustine in dissolved or solubilized form and are intended to be used as such or upon further dilution in intravenous diluents.

The pharmaceutical formulation of the present invention is a liquid injectable formulation that is free of propylene glycol. The inventors have surprisingly found that the presence of water and aminoacids aid in making the formulation stable.

In one preferred embodiment, the formulations of the present invention comprise Bendamustine, amino acids, suitable solvents such as polyethylene glycol, glycerol, water, ethanol and optionally other pharmaceutically acceptable excipients. Suitable amino acids include, but not limited to glycine, histidine, arginine, alanine and lysine.

The following table outlines the experiments carried out to determine the suitability of excipients in the formulation. It is to be noted that the experimental results are not meant to be interpreted as limiting the scope of the invention. The details are tabulated in table 1.

TABLE 1
Effect of excipients on impurity levels

| | Quantity per ml. | |
|---|---|---|
| Ingredients | Trial-1 | Trial-2 |
| Bendamustine Hydrochloride | 90 mg | 90 mg |
| Polyethyene glycol 400 | 890 mg | 863 mg |
| Glycerol | 157 mg | 183 mg |
| Monothioglycerol | — | 5 mg |
| L-Arginine | 1.0 mg | — |
| Water for injection | 10.0 mg | — |

Observations

| | | |
|---|---|---|
| Total impurities % w/w (at 60° C. for 12 hrs) | 0.45 | 4.54 |

From table 1, it is evident that the formulation comprising amino acid and water resulted in a stable product. Total impurities were significantly reduced in the formulation containing amino acid and water which was surprising.

Suitable solvents can be selected from, but are not limited to, polyethylene glycol, glycerol, water, ethanol, dimethyl sulfoxide (DMSO), 1-methyl-2-pyrrolidone (NMP), 1,3-dimethyl-2-imidazolidinone (DMI), acetone, tetrahydrofuran (THF), dimethylformamide (DMF), propylene carbonate (PC), dimethyl isosorbide and mixtures thereof. Preferred solvents are polyethylene glycol, glycerol, water and ethanol.

The pharmaceutical formulations of the present invention may also contain one or more anti-oxidants, preservatives, complexing agents and chelating agents such as, but are not limited to butylated hydroxyanisole (BHA), butylated hydroxyl toluene (BHT), citric acid, lactic acid, benzoic acid, tocopherol (Vitamin E), monothioglycerol, ascorbic acid, methyl paraben, benzyl alcohol, propyl gallate, surfactants, lipids, thioglycolic acid, niacinamide, nicotinic acid, creatine, cyclodextrins; ethylene diamine tetraacetic acid (EDTA), diethylenetriaminepentaacetic acid (DTPA), ethylene glycol-bis(β-aminoethyl ether)-tetraacetic acid (EGTA), N (hydroxy ethyl) ethylenediaminetriacetic acid (HEDTA), nitrilotriacetic acid (NTA), triethanolamine, 8-hydroxyquinoline, tartaric acid, phosphoric acid, gluconic acid, thiodipropionic acid, acetonic dicarboxylic acid, lecithin, di(hydroxyethyl)glycine, sorbitol or its pharmaceutically acceptable salts thereof. The formulation of the present invention optionally contains additional stabilizers.

The pharmaceutical formulation may optionally contain buffers such as citrate, glutamate, bicarbonate, tartrate, benzoate, lactate, gluconate, TRIS buffer, acetate buffer, boric acid buffer, phosphate buffer, meglumine, or any other suitable buffer.

Formulations of the present invention comprise pharmaceutically useful concentrations of Bendamustine, or a pharmaceutically acceptable salt thereof. The concentrations may range from about 25 mg/mL to about 200 mg/mL.

Bendamustine formulations prepared according to the invention were tested for stability. Another preparation with quantitative composition of Treanda® (liquid formulation) was prepared for a comparative stability study. The stability data of the invention formulation with the reference product is summarized in Table 2. It is surprisingly found that the use of amino acids in combination with suitable solvents yields stable product. No impurities were observed at relative retention time (RRT 1.05) for the invention product.

TABLE 2
Comparative stability data of invention product of example-4 with reference product

| | Condition | | | | | | | |
|---|---|---|---|---|---|---|---|---|
| | 1 M | 3 M | 1 M | 3 M | 1 M | 3 M | 1 M | 3 M |
| | 2-8° C. | | 25° C./60% RH | | 2-8° C. | | 25° C./60% RH | |
| | Invention Product | | | | Reference Product | | | |
| pH | 3.51 | 3.96 | 3.57 | 3.88 | 3.08 | 2.68 | 3.14 | 2.78 |
| Osmolality (mOsm/kg) | 301 | 309 | 310 | 303 | 365 | 378 | 368 | 381 |
| Assay (%) | 102.6 | 99.5 | 101.3 | 96.5 | 100.4 | 98.2 | 98.6 | 94.0 |
| Related substances | Impurities % (w/w) | | | | | | | |
| Deschloroethyl Bendamustine | 0.03 | 0.04 | 0.06 | 0.15 | 0.05 | 0.18 | 0.27 | 0.53 |
| HP-1 | 0.05 | 0.11 | 0.13 | 0.28 | 0.12 | 0.16 | 0.13 | 0.16 |
| HP-1 Ester | 0.02 | 0.05 | 0.16 | 0.51 | ND | 0.03 | ND | 0.02 |
| Bendamustine dimer | 0.03 | 0.05 | 0.11 | 0.32 | ND | ND | ND | ND |
| (unhydrolysed) | | | | | | | | |
| Unknown @ RRT 1.05 | ND | ND | ND | ND | 0.08 | 0.39 | 0.74 | 2.07 |
| Total Impurities | 0.16 | 0.34 | 0.78 | 2.12 | 0.40 | 0.90 | 1.4 | 3.4 |

ND: Not Detected

The following examples further describe certain specific aspects and embodiments of the present invention and demonstrate the practice and advantages thereof.

### Example 1

| S. No | Ingredients | Quantity/mL |
|---|---|---|
| 1 | Bendamustine hydrochloride | 70.0 mg |
| 2 | Polyethylene glycol 400 | 907 mg |
| 3 | Glycerol | 160 mg |
| 4 | Monothioglycerol | 5.00 mg |
| 5 | L-Arginine | 1.00 mg |
| 6 | Water for injection | 10.00 mg |

Manufacturing Process: Polyethylene glycol was taken in a compounding vessel and glycerol was added. Bendamustine hydrochloride was added and stirred, followed by the addition of monothioglycerol. The bulk solution was cooled to 2-8° C. L-Arginine dissolved in water for injection, was added to the above solution and stirred. The solution was filtered and filled into vials and stored at 2° C. to 8° C.

### Example 2

| S. No | Ingredients | Quantity/mL |
|---|---|---|
| 1 | Bendamustine hydrochloride | 90.0 mg |
| 2 | Polyethylene glycol 400 | 890 mg |
| 3 | Glycerol | 157 mg |
| 4 | Monothioglycerol | 5.00 mg |
| 5 | L-Arginine | 1.00 mg |
| 6 | Water for injection | 10.00 mg |

Manufacturing Process: Polyethylene glycol was taken in a compounding vessel and glycerol was added. Bendamustine hydrochloride was added and stirred, followed by the addition of monothioglycerol. The bulk solution was cooled to 2-8° C. L-Arginine dissolved in water for injection, was added to the above solution and stirred. The solution was filtered and filled into vials.

The filled vials were checked for stability at 60° C. for 12 hours. The total impurities were found to be around 0.4%.

### Example 3

| S. No | Ingredients | Quantity/mL |
|---|---|---|
| 1 | Bendamustine | 90.0 mg |
| | hydrochloride | |
| 2 | Polyethylene glycol 400 | 890 mg |
| 3 | Glycerol | 157 mg |
| 4 | L-Arginine | 1.00 mg |
| 5 | Water for injection | 10.00 mg |

Manufacturing Process: Polyethylene glycol was taken in a compounding vessel and glycerol was added. Bendamustine hydrochloride was added and stirred. The bulk solution was cooled to 2-8° C. L-Arginine dissolved in water for injection was added to the above solution and stirred. The solution was filtered and filled into suitable containers.

### Example 4

| S. No | Ingredients | Quantity/mL |
|---|---|---|
| 1 | Bendamustine | 50 mg |
| | hydrochloride | |
| 2 | Polyethylene glycol 400 | 893 mg |
| 3 | Glycerol | 158 mg |
| 4 | L-Arginine | 1 mg |
| 5 | Water for injection | 50 mg |

Manufacturing Process: Polyethylene glycol was taken in a compounding vessel and glycerol was added. Bendamustine hydrochloride was added and stirred. The bulk solution was cooled to 2-8° C. L-Arginine dissolved in water for injection was added to the above solution and stirred. Final volume of the solution was made up with polyethylene glycol 400. The solution was filtered and filled into suitable containers.

Bendamustine formulation prepared according to the invention was tested for stability at various conditions. The stability data of the invention formulation is summarized in Table 2. The product is tested for stability by storing at various conditions like 2-8° C. and 25° C.±60% RH for a period of 3 months.

We claim:

1: A stable, liquid formulation of Bendamustine comprising
(i) Bendamustine or its pharmaceutically acceptable salts, solvates, hydrates thereof
(ii) one or more aminoacids
(iii) one or more solvents and
(iv) optionally other pharmaceutically acceptable excipients,
wherein the formulation is free of propylene glycol.

2: The formulation of Bendamustine of claim 1, comprising
(i) Bendamustine hydrochloride
(ii) one or more aminoacids selected from arginine, alanine, glycine, histidine and lysine or its salts
(iii) one or more solvents and
(iv) optionally other pharmaceutically acceptable excipients,
wherein the formulation is free of propylene glycol.

3: The formulation of claim 2, wherein one or more solvents are selected from polyethylene glycol, glycerol, water and ethanol.
